(19) United States
(12) Patent Application Publication
(10) Pub. No.: US 2018/0313856 A1
(43) Pub. Date: Nov. 1, 2018

(54) SERUM LIPID BIOMARKERS OF PREECLAMPSIA

(71) Applicant: Brigham Young University, Provo, UT (US)

(72) Inventors: Steven William GRAVES, Highland, UT (US); Michael Sean ESPLIN, Salt Lake City, UT (US)

(73) Assignee: Brigham Young University, Provo, UT (US)

(21) Appl. No.: 15/771,174

(22) PCT Filed: Oct. 26, 2016
(86) PCT No.: PCT/US2016/058824

§ 371 Date: Apr. 26, 2018

Related U.S. Application Data
(60) Provisional application No. 62/246,500, filed on Oct. 26, 2015.

(51) Int. Cl. G01N 33/92

(52) U.S. Cl. CPC G01N 33/92; G01N 2800/50; G01N 2800/368

(57) ABSTRACT

A panel of lipid biomarkers of preeclampsia in pregnant women, useful prior to onset of symptoms to identify women at risk of preeclampsia as early as 12-14 weeks gestation is disclosed. Methods of diagnosing a pregnant subject at risk of developing preeclampsia, and methods of reducing symptoms or preventing the onset of preeclampsia in a pregnant subject, employing the disclosed lipid biomarkers are also disclosed.

### RELATED APPLICATIONS

This application claims the benefit under 35 U.S.C. § 119(e) of Provisional U.S. Patent Application No. 62/246,500, filed Oct. 26, 2015, the contents of which are hereby incorporated by reference, in their entirety.

### BACKGROUND

### 1. Technical Field

This application relates to biomarkers for preeclampsia in pregnant women. More particularly, this application relates to a panel of lipid markers that are predictive of increased risk of developing preeclampsia before symptoms occur, methods for using such lipid markers, and methods of reducing symptoms or preventing onset of preeclampsia in a pregnant subject.

### 2. Background Information

Preeclampsia (PE) is a potentially life-threatening disorder of pregnancy characterized by new-onset hypertension and proteinuria after 20 weeks gestation. PE constitutes a leading cause of maternal and perinatal mortality and morbidity (ACOG Practice Bulletin; Higgins et al.). Estimates are that as many as 75,000 women worldwide die each year from PE (Bellamy et al.).

The specific causes of PE are still debated and have yet to be established. Treatment options are very limited and frequently require early termination of the pregnancy, regardless of gestational age, accounting for about 20% of all preterm births (Goldenberg et al) Infants born to preeclamptic mothers may be at increased risk of hypertension, heart disease and diabetes (Barker et al.).

The pathogenesis of PE is complex but it is generally believed that one or more very early events in pregnancy contribute to PE. One theory involves an incomplete remodeling of maternal spiral arteries by invasive extravillous placental trophoblast cells, resulting in inadequate perfusion of the fetal-placental unit with attendant ischemia (Hassan et al.; Cnossen et al.). Abnormal waveform patterns and an increased pulsatility index observed in uterine Doppler ultrasound studies support the concept of an underperfused fetus prior to clinically-apparent PE (Espinosa et al.; Cnossen et al.). There is also evidence of very early biochemical changes in women who develop PE later in the same pregnancy. Other biochemical changes appear to precede clinically-evident PE by a few weeks, including hypoxia-reoxygenation (Hung et al.), abnormal expression of angiogenic and anti-angiogenic factors in the maternal circulation, and endothelial dysfunction with a pro-inflammatory response (Maynard et al.; Roberts et al.). However, to date there are still no accepted, predictive biomarkers of PE (Kleinrouweler et al.; Powers et al.; Chaiworapongsa et al.).

It is now possible to survey hundreds to thousands of molecules in biological specimens in an unbiased way using mass spectrometry (MS). Proteomics is the most common of these approaches and has been employed to study several diseases, but its use to explore diagnostic or predictive biomarkers in serum is difficult, due to ˜30 highly abundant serum proteins that mask the vast majority of lower abundance species in serum.

Earlier identification of women at risk for developing preeclampsia will allow clinicians to reduce or eliminate many complications of the disease, saving lives. It would be particularly useful to identify markers in serum that can be evaluated before symptoms present.

### BRIEF SUMMARY

In one aspect, a panel of lipid markers of preeclampsia is disclosed, including at least three lipid markers selected from the group having a mass to charge ratio of about 263.2, 383.3, 445.4, 462.3, 645.5, 784.6, 796.6, 798.6, 810.6, 836.6, 916.8, 920.8, and combinations thereof.

In some embodiments, the serum sample is from a pregnant subject at about 12-14 weeks gestation.

In some embodiments, pregnant subjects having elevated levels of at least one lipid marker selected from the group having a mass to charge ratio of about 263.2, 383.3, 462.3, 734.6, 760.6, 784.6, 796.6, 798.6, 810.6, 836.6, and combinations thereof, as compared to normal pregnant subjects of about the same gestational age, have an elevated risk of preeclampsia.

In some embodiments, pregnant subjects having decreased levels of at least one lipid marker selected from the group having a mass to charge ratio of about 301.2, 425.1, 445.4, 645.5, 714.6, 788.6, 895.7, 916.8, 920.8, 928.8, 954.8, 956.8, 958.8, and combinations thereof, as compared to normal pregnant subjects of about the same gestational age, have an elevated risk of preeclampsia.

In one aspect, methods are provided for diagnosing a pregnant subject as having preeclampsia or being at increased risk for preeclampsia, including analyzing a serum sample from a pregnant subject for levels of one or more lipid markers; and diagnosing the pregnant individual as having preeclampsia or being at increased risk for preeclampsia based on levels of the lipid markers relative to levels in a normal pregnant subject of about the same gestational age.

In embodiments, the serum sample is analyzed for levels of any one two, three, four, five, six, seven, eight, nine, ten, eleven, or twelve lipid markers selected from the group having a mass to charge ratio of about 263.2, 383.3, 445.4, 462.3, 645.5, 784.6, 796.6, 798.6, 810.6, 836.6, 916.8, 920.8.

In one aspect, methods are disclosed for reducing symptoms or preventing the onset of preeclampsia in a pregnant subject.

In some embodiments, the method includes sampling serum from a pregnant individual at about 12-14 weeks gestation; analyzing the sample for levels of three or more lipid markers in the serum; comparing the levels of the three or more lipid markers to levels in control subjects of about the same gestational age; and determining a clinical treatment to reduce symptoms or prevent onset of preeclampsia in the pregnant subject.

In some embodiments, the three or more lipid markers are each separately selected from the group having a mass to charge ratio of about 263.2, 383.3, 445.4, 462.3, 645.5, 784.6, 796.6, 798.6, 810.6, 836.6, 916.8, 920.8, and combinations thereof.

### BRIEF DESCRIPTION OF THE DRAWINGS

FIG. 1 shows two area-under-the-curve plots. FIG. 1a is the resulting receiver operator characteristic curve generated by logistic regression analysis that included 6 lipid biomarkers having mass to charge ratios of 383, 445, 784, 796, 798 and 920.

FIG. 1b is the resulting receiver operator characteristic curve generated by logistic regression analysis of another combination of 6 lipid biomarkers having mass to charge ratios of 263, 383, 445, 645, 784 and 916. Some peaks are common to the two panels.

### DETAILED DESCRIPTION

Disclosed herein are various exemplary embodiments of the invention. The following embodiments are not meant to limit the invention or narrow the scope thereof. It will be apparent to one of ordinary skill in the art that suitable modifications and adaptations may be made without departing from the scope of the invention. All patents and publications cited herein are incorporated by reference for the specific teachings thereof.

Terms such as “include,” “including,” “contain,” “containing,” “has,” or “having” and the like mean “comprising.” Headings herein are provided for the convenience of the reader and are not intended to limit the scope of the subject matter described thereafter.

Despite decades of research, early diagnosis of preeclampsia (PE) remains elusive. While there are known risk factors for PE, it is not yet possible to precisely distinguish pregnancies destined to develop PE from those that will not. This has made prospective clinical studies large and expensive.

The applicants have identified novel lipid biomarkers of PE, and methods of using such lipid biomarkers to predict the risk of PE in a pregnant subject. These lipid biomarkers are useful in a clinical program to reduce symptoms or prevent the onset of preeclampsia in pregnant subjects.

As used herein, the term “biomarker” may be used to refer to a naturally-occurring biological molecule present in pregnant women at varying concentrations useful in predicting the risk of preeclampsia and related uses. For example, the biomarker can be a lipid present in higher or lower amounts in a subject at risk of developing preeclampsia relative to the amount of the same biomarker in a subject who did not develop preeclampsia during pregnancy.

As used herein, the terms “lipid marker” and “lipid biomarker” may be used interchangeably to refer to lipid-based or lipid-like biochemicals present in body tissues, particularly in body tissues of a pregnant female that may be useful in predicting the risk of preeclampsia and related uses. Non-limiting examples of classes of lipid markers according to the present disclosure include cholesterol; cholesterol derivatives such as cholesterol esters, ketocholesterols, and hydroxycholesterols; fatty acids, fats/triglycerides/triacylglycerols; and phosphocholines/phosphatidyl cholines.

As used herein, the term “subject” refers to a pregnant woman at risk of developing preeclampsia who may benefit from the methods described herein.

Lipids are increasingly recognized as having important biological roles or representing important biochemical correlates of clinical changes. A wide variety of human diseases are associated with aberrant lipid metabolism including Alzheimer's disease, diabetes and atherosclerosis (Wenk; Watson; Steinberg). Alterations in lipids may represent by-products of underlying pathophysiology but could also represent primary disease mediators. For example, arachidonic acid is a precursor for eicosanoids which have a significant role in inflammatory processes. There is evidence of changes in downstream products of arachidonic acid in PE (Balazy). As another example, oxidized lipid species can reflect increased reactive oxygen species (ROS) which are frequently produced as a consequence of some diseases (Butterfield et al.; Suarna et al.; Berliner et al.). Therefore, lipid profiles in pregnant women at an early stage may be informative using lipidomic biomarkers to identify patients at significant risk for PE.

‘Shotgun’, i.e. global, in-depth or comprehensive lipidomics can survey thousands of unique lipids in a single biological specimen. One such lipidomic approach using direct injection, electrospray ionization coupled with highly mass accurate, mass spectrometers (ESI-MS) represents a powerful tool for cataloguing and quantifying lipids in tissue, cells or body fluids. Lipidomics can complement peptidomic and proteomic methods. Lipidomics, especially direct injection lipidomics, is substantially less involved than top-down, global proteomic methods, which typically require enzyme digestion and multiple separation steps prior to MS.

Consequently, a ‘global’ serum lipidomic approach, involving lipid extraction followed by direct injection, time-of-flight mass spectrometry was used to identify and chemically characterize predictive serum PE lipid biomarkers. We hypothesized that this approach would find individual lipids and sets of lipids that would allow for the prediction of a substantial portion of women would later develop PE in the same pregnancy and that the changes would provide insights into the mechanisms early in the development of PE.

Thus, in one aspect, the disclosure provides one more lipid markers of PE.

In some embodiments, the lipid markers include one or more lipid markers selected from the group of lipid markers having a mass to charge ratio of about 263.2, 383.3, 445.4, 462.3, 645.5, 784.6, 796.6, 798.6, 810.6, 836.6, 916.8, 920.8, and combinations thereof, as determined by electrospray injection time-of-flight mass-spectrometry.

As used herein, and with respect to the mass to charge ratio of lipid markers in various embodiments disclosed herein, the term “about”—unless otherwise specified—means a mass to charge ratio that reflects the indicated value +/−0.1 where the value is reported with one significant figure beyond the decimal point (e.g., for discovery studies and validation studies disclosed herein). The term “about” means a mass to charge ratio that reflects the indicated value +/−0.03 where the value is reported with two significant figures beyond the decimal point (e.g., for the characterization studies disclosed herein), reflecting the different calibrations used for different studies. For example, it is understood that “about 462.3” shall mean “462.3+/−0.1”, including all values between 462.2 and 462.4, inclusive; while “about 462.30” shall mean “462.30+/−0.03”, including all values between 462.27 and 462.33, inclusive.

While some or all of the disclosed biomarkers may be present in most pregnant women, many pregnant women that go on to experience preeclampsia have either higher or lower blood serum concentrations of one or more of these biological molecules during pregnancy as compared to women that had normal births, as provided further herein. For example, the lipid marker having a mass to charge ratio of about 263.2 may be significantly more abundant in PE cases while the lipid marker having a mass to charge ratio of about 920.8 may be significantly more abundant in controls. Thus a comparison of the abundance of one or more of these biomarkers in a biological sample from a subject against a known control concentration from subjects that did not experience preeclampsia, or against a known biomarker concentration from the subject being tested, may be predictive of such complications.

Those subjects having a higher or lower abundance of one or more of these biomarkers may have an increased risk of preeclampsia, and can thus be identified early enough to allow appropriate treatment. The abundance of particular biomarkers useful for predicting preeclampsia is described in detail below.

Where one biomarker may have predictive value and value in determining a clinical intervention or treatment of preeclampsia, multiple markers used in combination may be still more useful, increasing sensitivity and accuracy of the prediction.

Thus, in some embodiments, a panel of lipid markers of PE is provided.

As used herein, the term “panel” of lipid markers refers to a group of lipid markers that includes at least two such lipid markers in combination. For example, a panel of lipid markers of PE may include 2, 3, 4, 5, 6, 7, 8, 9, 10, 11, 12 or more lipid markers that are associated with the development of PE, particularly in a human pregnant female.

In some embodiments, a panel of lipid markers of preeclampsia is provided, the panel including at least two lipid markers selected from the group having a mass to charge ratio of about 263.2, 383.3, 445.4, 462.3, 645.5, 784.6, 796.6, 798.6, 810.6, 836.6, 916.8, 920.8, and combinations thereof, as determined by electrospray injection time-of-flight mass-spectrometry.

In a specific embodiment, the panel of lipid markers of preeclampsia includes at least three lipid markers selected from the group having a mass to charge ratio of about 263.2, 383.3, 445.4, 462.3, 645.5, 784.6, 796.6, 798.6, 810.6, 836.6, 916.8, 920.8.

In a specific embodiment, the panel of lipid markers of preeclampsia includes at least four lipid markers selected from the group having a mass to charge ratio of about 263.2, 383.3, 445.4, 462.3, 645.5, 784.6, 796.6, 798.6, 810.6, 836.6, 916.8, 920.8.

In a specific embodiment, the panel of lipid markers of preeclampsia includes at least five lipid markers selected from the group having a mass to charge ratio of about 263.2, 383.3, 445.4, 462.3, 645.5, 784.6, 796.6, 798.6, 810.6, 836.6, 916.8, 920.8.

In a specific embodiment, the panel of lipid markers of preeclampsia includes at least six lipid markers selected from the group having a mass to charge ratio of about 263.2, 383.3, 445.4, 462.3, 645.5, 784.6, 796.6, 798.6, 810.6, 836.6, 916.8, 920.8.

In a specific embodiment, the panel of lipid markers of preeclampsia includes at least three seven lipid markers selected from the group having a mass to charge ratio of about 263.2, 383.3, 445.4, 462.3, 645.5, 784.6, 796.6, 798.6, 810.6, 836.6, 916.8, 920.8.

In a specific embodiment, the panel of lipid markers of preeclampsia includes at least eight lipid markers selected from the group having a mass to charge ratio of about 263.2, 383.3, 445.4, 462.3, 645.5, 784.6, 796.6, 798.6, 810.6, 836.6, 916.8, 920.8.

In a specific embodiment, the panel of lipid markers of preeclampsia includes at least nine lipid markers selected from the group having a mass to charge ratio of about 263.2, 383.3, 445.4, 462.3, 645.5, 784.6, 796.6, 798.6, 810.6, 836.6, 916.8, 920.8.

In a specific embodiment, the panel of lipid markers of preeclampsia includes at least ten lipid markers selected from the group having a mass to charge ratio of about 263.2, 383.3, 445.4, 462.3, 645.5, 784.6, 796.6, 798.6, 810.6, 836.6, 916.8, 920.8.

In a specific embodiment, the panel of lipid markers of preeclampsia includes at least eleven lipid markers selected from the group having a mass to charge ratio of about 263.2, 383.3, 445.4, 462.3, 645.5, 784.6, 796.6, 798.6, 810.6, 836.6, 916.8, 920.8.

In a specific embodiment, the panel of lipid markers of preeclampsia includes twelve lipid markers selected from the group having a mass to charge ratio of about 263.2, 383.3, 445.4, 462.3, 645.5, 784.6, 796.6, 798.6, 810.6, 836.6, 916.8, 920.8.

In specific embodiments, the panel of lipid markers includes lipid markers having a mass to charge ratio of about 383.3, 645.5, and 784.6.

In specific embodiments, the panel of lipid markers includes lipid markers having a mass to charge ratio of about 383.3, 645.5, 784.6, and 263.2.

In specific embodiments, the panel of lipid markers includes lipid markers having a mass to charge ratio of about 383.3, 645.5, 784.6, 263.2, and 836.6.

The lipid markers disclosed herein may be described in various ways, including mass to charge ratio (ESI-TOF MS), chemical class (e.g., cholesterol esters), elemental composition, or specific chemical structure (which may be tentative).

In some embodiments, the disclosed lipid marker has an elemental composition selected from the group consisting of C₁₈H₃₀O+H⁺, C₂₀H₂₈O₂+H⁺, C₂₇H₄₃O+, C₂₉H₄₈O₃+H⁺, C₄₅H₇₂O₂+H⁺, C₄₉H₇₆O₂+NH₄+, C₄₀H₈₀NO₈P+H⁺, C₄₂H₈₂NO₈P+H⁺, C₄₄H₈₂NO₈P+H⁺, C₄₄H₈₆NO₈P+H⁺, C₄₆H₈₆NO₇P+H⁺, C₄₄H₈₀NO₉P+H⁺, C₄₆H₈₄NO₈P+H⁺, C₄₈H₈₆NO₈P+H⁺, C₅₂H₉₉N₂O₇P+H⁺, C₅₇H₁₀₂O₇+NH₄⁺, C₅₅H₉₈O₉+NH₄⁺, C₅₇H₉₈O₈+NH₄⁺, C₅₉H₁₀₄H₈+NH₄⁺, C₅₉H₁₀₂O₈+NH₄⁺, C₅₈H₁₀₀O₉+NH₄⁺, and combinations thereof.

In some embodiments, the disclosed lipid marker is selected from the group consisting of 7-keto cholesterol, C18:4 cholesterol ester, C22:6 cholesterol ester, PC-16:0/16:0, PC-16:0/18:1, PC-18:1/18:2, PC-18:0/18:1, PC-O-18:0/20:4, PC-(16:0/18:4)+OH, PC-18:0/20:4, PC-18:0/22:5, TG-18:0/18:2/18:2+OH, TG-18:2/16:0/18:2+OH+OOH, TG-18:1/18:1/18:2+OH+OH, TG-904.75 (18:1/18:2/20:4)+OH+OH, TG-906.77 (18:0/18:2/20:4)+OH+OH, TG-906.77 (18:1/18:1/20:4)+OH+OH, and combinations thereof.

Additional and specific panels are further disclosed herein, including Table 5 hereto.

Lipid biomarkers of the present disclosure may be measured in different biological samples, preferably biological fluids such as serum, plasma, or blood. Lipid biomarkers may also be assessed in tissues and/or in other biological fluid samples, including but not limited to amniotic fluid, cervical-vaginal fluid, synovial fluid, lavage fluid, urine, cerebrospinal fluid, tears, and saliva. If desired, a sample can be prepared to enhance detectability of the lipid biomarkers. For example, a serum sample can be fractionated, purified, or filtered. Any method that enriches for a biomarker lipid can be used.

The disclosed lipid markers are particularly suitable for early detection and/or early prediction of preeclampsia in a pregnant subject. The disclosed lipid biomarkers may thus be useful in a period before clinical symptoms of PE have developed.

In embodiments, the lipid markers are obtained from pregnant subjects at about 10-20 weeks gestation. In some embodiments, the lipid markers are obtained from serum of pregnant subjects at about 12-14 weeks gestation.

The lipid biomarkers may be isolated lipid biomarkers.

As used herein, the term “isolated,” with respect to lipids, refers to material that has been removed from its original environment, if the material is naturally occurring. For example, a naturally-occurring lipid present in a female subject is not isolated, but the same lipid, which is separated from some or all of the coexisting materials in the natural system, is isolated. Such an isolated lipid could be part of a composition and still be isolated in that the composition is not part of its natural environment.

In one aspect, methods are provided for diagnosing a pregnant subject as having preeclampsia or being at increased risk for preeclampsia.

In some embodiments, the method may include analyzing a serum sample from a pregnant subject for levels of at least one lipid marker and diagnosing the pregnant individual as having preeclampsia or being at increased risk for preeclampsia based on levels of the lipid markers relative to levels in a normal pregnant subject of about the same gestational age.

In some embodiments, the serum sample is obtained from a pregnant subject at about 12-14 weeks gestation. The at least one lipid marker may be detected at least 3 to 6 months prior to presentation of a clinical symptom associated with preeclampsia. PE is normally diagnosed after 20 weeks gestation. The disclosed methods may thus provide valuable information regarding still-asymptomatic patients who are nevertheless at risk of developing PE.

In embodiments, the at least one lipid marker is selected from the group having a mass to charge ratio of about 263.2, 301.2, 383.3, 425.1, 445.4, 462.3, 645.5, 714.6, 734.6, 760.6, 784.6, 788.6, 796.6, 798.6, 810.6, 836.6, 895.7, 916.8, 920.8, 928.8, 954.8, 956.8, 958.8, and combinations thereof, as determined by electrospray injection time-of-flight mass-spectrometry (ESI-TOF MS).

In some embodiments, the method includes analyzing the serum sample for levels of two, three, four, five, six, seven, eight, nine, ten, eleven, or twelve lipid markers selected from the group having a mass to charge ratio of about 263.2, 383.3, 445.4, 462.3, 645.5, 784.6, 796.6, 798.6, 810.6, 836.6, 916.8, and 920.8.

Other specific panels useful for the disclosed methods are disclosed in, for example, Table 5 hereto.

As disclosed herein, increased or decreased levels of particular lipid biomarkers, as compared to normal pregnant subjects of about the same gestational age, are predictive of increased risk of developing preeclampsia. Alternatively, increased or decreased levels of particular lipid biomarkers are indicative of preeclampsia in either the clinical or pre-clinical stage.

For example, in some embodiments, pregnant subjects having elevated levels of at least one lipid marker selected from the group having a mass to charge ratio of about 263.2, 383.3, 462.3, 734.6, 760.6, 784.6, 796.6, 798.6, 810.6, 836.6, and combinations thereof, as compared to normal pregnant subjects of about the same gestational age, have an elevated risk of preeclampsia.

Similarly, in some embodiments, pregnant subjects having decreased levels of at least one lipid marker selected from the group having a mass to charge ratio of about 301.2, 425.1, 445.4, 645.5, 714.6, 788.6, 895.7, 916.8, 920.8, 928.8, 954.8, 956.8, 958.8, and combinations thereof, as compared to normal pregnant subjects of about the same gestational age, have an elevated risk of preeclampsia.

The disclosed methods are very sensitive at diagnosing a pregnant subject as having preeclampsia or being at increased risk for preeclampsia. In some embodiments, the pregnant subject exhibits at least 80% sensitivity for the lipid biomarkers.

In one aspect, the further provides methods of reducing symptoms or preventing the onset of preeclampsia in a pregnant subject.

In embodiments, the method includes sampling serum from a pregnant individual at about 12-14 weeks gestation; analyzing the sample for levels of three or more lipid markers in the serum; and comparing the levels of the three or more lipid markers to levels in control subjects of about the same gestational age; and determining a clinical treatment to reduce symptoms or prevent onset of preeclampsia in the pregnant subject.

In some embodiments, the three or more lipid markers are each separately selected from the group having a mass to charge ratio of about 263.2, 383.3, 445.4, 462.3, 645.5, 784.6, 796.6, 798.6, 810.6, 836.6, 916.8, 920.8, and combinations thereof, as determined by electrospray injection time-of-flight mass-spectrometry.

In some embodiments, the method includes analyzing the serum sample for levels of two, three, four, five, six, seven, eight, nine, ten, eleven, or twelve lipid markers selected from the group having a mass to charge ratio of about 263.2, 383.3, 445.4, 462.3, 645.5, 784.6, 796.6, 798.6, 810.6, 836.6, 916.8, and 920.8.

Other specific panels useful for the disclosed methods are disclosed in, for example, Table 5 hereto.

In some embodiments, the method further includes treating the pregnant subject for preeclampsia. For example, pregnant subjects determined to have increased risk of preeclampsia according to the disclosed methods may be identified as high-risk patients for regular monitoring and early intervention. This may involve, for example, early administration of a suitable medicament, therapy, isolation, rest, or nutritional intervention. Treatment in this early stage may be effective for alleviating, or preventing onset of, PE.

### Example

Overview

Shotgun lipidomic studies were undertaken to determine if predictive serum lipid biomarkers exist that can differentiate future cases from controls at an early stage of pregnancy. A discovery study was performed using sera collected at 12-14 weeks of pregnancy from 27 controls with uncomplicated pregnancies and 29 cases that later developed PE. Lipids were extracted using organic solvent and analyzed by direct infusion into a time-of-flight mass spectrometer (TOF-MS). Peaks demonstrating apparent differences were selected, their abundances determined and statistical differences tested. Statistically-significant lipid markers were reevaluated in a second confirmatory study having 43 controls and 37 PE cases. Multi-marker combinations were developed using lipid biomarkers confirmed in the second set. The initial study detected 45 potential PE markers. Of these, 23 markers continued to be statistically significant in the second confirmatory set.

Most of these markers representing different lipid classes were identified using tandem MS studies. Several multi-marker panels with AUC >0.85 and high predictive values were developed. Developed panels of serum lipidomic biomarkers appear able to identify most women at risk for PE in a given pregnancy at 12-14 weeks gestation.

### Methods

Study Population

Serum specimens used for both the discovery and confirmatory sets were obtained. All samples were banked sera, obtained from a previously completed clinical study. Sera were collected from pregnant women at 12-14 weeks gestation who were followed through the completion of their pregnancies. Specimens were analyzed without clinical identifiers.

A discovery cohort involved sera from 27 controls having term, uncomplicated pregnancies and 29 cases, which developed PE later during the pregnancy. The second confirmatory study of the promising potential lipid biomarkers from the discovery cohort involved 37 cases and 43 controls, also collected at 12-14 weeks gestation. Demographic characteristics are summarized in Tables 1a and 1b below.

TABLE 1a
Demographics for First, Discovery Study

| | | Gestational | | | | Baby | |
|---|---|---|---|---|---|---|---|
| | Maternal | age at admit | BMI | | Diastolic | weight | |
| | age (yrs) | (wks) | (kg/m²) | Systolic BP | BP | (gm) | Race |
| Cases | 30 ± 4.6 | 36.4 ± 3.07 | 30.8 ± 5.42 | 149.5 ± 17.4 | 90.1 ± 12.3 | 2755 ± 801.1 | 1/29-Black |
| | | | | | | | 28/29-White |
| Controls | 27 ± 4.8 | 39 ± 1.23 | 30.4 ± 5.6 | 138.6 ± 14.7 | 80 ± 12.5 | 3459 ± 348.6 | 27/27-White |

TABLE 1b
Demographics for Second, Confirmatory Study.

| | | Gestational | | | | Baby | |
|---|---|---|---|---|---|---|---|
| | Maternal | age at admit | BMI | | Diastolic | weight | |
| | age (yrs) | (wks) | (kg/m²) | Systolic BP | BP | (gm) | Race |
| Cases | 29 ± 4.7 | 36.3 ± 3.2 | 31.7 ± 5.89 | 147 ± 20.8 | 90 ± 15.1 | 2817 ± 860.9 | 37/37-White |
| Controls | 26 ± 4.7 | 38.7 ± 1.6 | 27.9 ± 4.7 | 117 ± 14.4 | 70.4 ± 8.2 | 3295 ± 535.2 | 43/43-White |

Sample Preparation.

All specimens were stored at −80° C. on dry ice and were maintained at −80° C. before and after sample processing. Serum lipid extraction efficiency was tested using 5 different organic extraction solutions. The applicants employed a modified, previously-described extraction protocol that involved a solvent mixture of hexane:isopropanol (3:2) (Hara et al.). To 200 μL of serum, 1.8 mL hexane:isopropanol (3:2) and 300 μL of 0.5 M KH₂PO₄ were added in a glass tube followed by vigorous vortexing. Samples were further agitated on a shaking platform for 1 h at room temperature at the speed of 80 rpm. To complete the extraction, 150 μL of water were added, mixed and centrifuged at 2000 rpm for 12 min. The upper, organic phase containing lipids was collected and dried completely under nitrogen. Dried lipid extracts were redissolved in 200 μL of chloroform:methanol (3:1) and stored at −80° C.

MS Analysis of Lipid Extracts

To a 20 μL aliquot of the sample extract, 23 μL of chloroform, 46 μL of methanol, and 14 μL of 12 μM ammonium acetate were added. The samples were directly injected into the mass spectrometer (6230 TOF LC/MS Agilent Technologies) through electrospray ionization (ESI) source operated in the positive ion mode. A syringe pump was utilized to inject samples at the flow rate of 2 μL/min. The ESI source used a microspray needle having an i.d. of 50 μm. The capillary voltage was set at 3500 V. MS data was collected from mass to charge ratios (m/z) 100-3000 with an acquisition rate of 1 spectrum/sec.

Nebulizer gas and dry gas parameters were optimized to obtain a stable flow. The dry gas was set to 5 L/min at 325° C. with a nebulization gas pressure of 1.03 bar. Mass Hunter-Qualitative software [Agilent Technologies] was used for data analysis. Each specimen generated a mass spectrum from which the total ion chromatogram (TIC the sum of all ion counts) from m/z 100 to 3000 was determined. A peak list having m/z values for all peaks with their abundances was generated from the mass spectrum.

To reduce analytical variability, all MS peaks were normalized. For normalization, 7 abundant peaks representing different classes of lipids but showing similar abundance in both case and control sera (p>0.40) were chosen as a reference set and their abundances averaged. The mass-to charge ratios (m/z) and classes for these peaks are as follows: m/z 203 (fatty acid), 369 (sterol), 666 (cholesterol ester), 758 (phosphatidyl choline (PC)), 782 (PC), 810 (PC) and 848 (triacyl glycerol). These peaks were consistently seen in all the samples. The ratio of the average intensity of these 7 peaks was comparable or more consistent than the use of TIC counts for peak normalization, but avoided the occasional high TIC for a few MS runs with a high background. Therefore, the average of the combined intensity of these 7 peaks was used to normalize all peaks across the each individual mass spectrum.

For the second confirmatory study, all samples were processed using the same method and were directly injected onto the same MS instrument at a flow rate of 10 μL/min, using a standard sprayer having an i.d. of 120 μm. All other parameters were kept identical. All the potential significant markers from the initial, discovery set were reanalyzed for their performance in the second confirmation set using Student's T-test.

Identification of Replicating Lipid Biomarkers

After candidate biomarkers were found and confirmed by ESI-TOFMS, tandem MS was performed to fragment and characterize all the validated biomarkers using ESI-quadrupole-TOFMS. Lipid markers were fragmented using either a QSTAR Pulsar 1 quadrupole (Applied Biosystems) or an Agilent 6530 Q-TOF MS in the positive ion mode. For the QSTAR, samples were injected directly at a flow rate of 2 μL/min. The capillary voltage was set to 4200 V. The selection of the mass range targeted for fragmentation depended upon the m/z of the parent compound with a spectral acquisition rate of 1 spectrum/sec. Declustering and focusing potentials were set to 65 V and 290 V respectively. Nitrogen and/or argon gas was used for collisionally-induced fragmentation. Multiple fragmentation energies were carried out to obtain as complete fragmentation as possible. MS/MS spectra were collected for 2 min and the multi-channel analyzer (MCA) function was turned on resulting in summation of all 120 MS/MS spectra together thus increasing signal to noise ratio.

For the Agilent 6530, sample injection was carried out at the flow rate of 10 μL/min. The capillary voltage was set to 3500 V. The drying gas flow rate and the temperature were 5 L/min and 3000 C. MS/MS spectra were collected from m/z 50-3000 and the spectral acquisition rate was 3 spectra/sec. Collision energies were optimized depending upon the parent ion to obtain maximum fragmentation coverage. Individual scans were summed using the add feature of the Mass Hunter program to obtain greater signal to noise. The targeted MS/MS mode was used to isolate and fragment the parent ion.

Exact mass studies were done using a set of internal standards. Lipidmaps.org was used to tentatively determine the lipid class from the different possible classes for a particular parent peak. Furthermore, product ions, neutral losses, fragmentation patterns and exact masses were compared with the observed fragmentation patterns to confirm the classes and identities of the markers.

Statistical Analyses

For comparisons between the cases and controls, the normalized intensities of all the peaks (abundance >200 ion counts) in the first study were subjected to a two-tailed Student's t-test. A p-value <0.05 calculated for each peak, as a single statistical comparison, was used as a threshold to the list of potential biomarkers. It is recognized that since the t-test statistic was performed for multiple peaks, separately, the true p-values are much larger than 0.05.

Consequently, any identified peak may not be statistically significant between the cases and controls. Using a one at a time p-value calculation with a minimum threshold, however, reduced the list of potential biomarkers. The list was augmented by adding peaks that had low p-values close to 0.05 but greater than this threshold, resulting in a list of 45 potential biomarkers. Those peaks that continued to show differential expression, as measured by a p-value of less than 0.05 as determined from a one at a time t-test in the second, independent set of specimens were considered highly likely to be biomarkers. Given the goal of generating panels of biomarkers, candidates with a p-value between 0.05 and 0.10 were also included because it is recognized that some markers may be selective for a subgroup within the broader diagnosis of PE and hence may be complementary to other markers. For this reason, they were considered in potential biomarker panels and were modeled to provide better sensitivities and specificities. Biomarker panel development employed a forward selection; leave one out, logistic regression analysis approach (Agresti; Devijver et al.). In modeling the MS data, there were a few peaks in a small number of specimens for which intensities could not be determined due either to peak masking by other nearby peaks or because of a sub-threshold abundance of a specific peak in that sample. To account for these missing values, the values for these peaks were estimated using multivariate imputation by chained equations. The method of chained equations used to predict the missing values was through predictive mean matching. This uses the values of the other samples to impute missing values, allowing for all peaks to still be used (Buuren et al.). Receiver operator characteristic curves for these panels were generated and allowed for the evaluation of sensitivity and specificity.

Results

Serum lipid PE biomarkers in a discovery study.

The applicants hypothesized that there would be serum lipid biomarkers present at 12-14 weeks gestation that are predictive of PE later in the same pregnancy. Using a global, serum lipidomic approach, the initial study of 27 controls and 29 cases identified 45 candidate markers that were statistically or near statistically different in women who developed PE compared with women who had uncomplicated pregnancies. The candidate markers are listed in Table 2.

TABLE 2
Candidate PE Lipid Biomarkers.

| No. | m/z | P value | Higher in |
|---|---|---|---|
| 1. | 211.0 | 0.1 | Cases |
| 2. | 228.2 | 0.07 | Cases |
| 3. | 239.1 | 0.06 | Cases |
| 4. | 256.2 | 0.08 | Cases |
| 5. | 257.1 | 0.07 | Cases |
| 6. | 263.2 | 0.08 | Cases |
| 7. | 280.9 | 0.04 | Controls |
| 8. | 282.2 | 0.08 | Cases |
| 9. | 301.2 | 7.1 × 10\hat{ }−6 | Controls |
| 10. | 383.3 | 2.5 × 10\hat{ }−8 | Cases |
| 11. | 425.1 | 0.08 | Controls |
| 12. | 430.2 | 0.05 | Cases |
| 13. | 462.3 | 0.05 | Cases |
| 14. | 445.4 | 3.4 × 10\hat{ }−4 | Controls |
| 15. | 520.3 | 0.08 | Cases |
| 16. | 531.4 | 0.02 | Cases |
| 17. | 548.4 | 0.05 | Cases |
| 18. | 642.6 | 0.03 | Cases |
| 19. | 645.5 | 0.06 | Controls |
| 20. | 663.5 | 2.5 × 10\hat{ }−5 | Cases |
| 21. | 714.6 | 0.009 | Controls |
| 22. | 734.6 | 0.07 | Cases |
| 23. | 760.6 | 0.08 | Cases |
| 24. | 774.5 | 0.04 | Cases |
| 25. | 784.6 | 0.05 | Cases |
| 26. | 894.7 | 0.02 | Cases |
| 27. | 788.6 | 0.06 | Cases |
| 28. | 796.6 | 0.09 | Cases |
| 29. | 798.6 | 0.03 | Cases |
| 30. | 810.6 | 0.1 | Cases |
| 31. | 836.6 | 0.1 | Cases |
| 32. | 838.6 | 0.07 | Cases |
| 33. | 895.7 | 0.02 | Controls |
| 34. | 896.7 | 0.02 | Controls |
| 35. | 898.8 | 0.02 | Controls |
| 36. | 904.8 | 0.08 | Controls |
| 37. | 916.8 | 0.01 | Controls |
| 38. | 920.7 | 0.009 | Controls |
| 39. | 922.7 | 0.001 | Controls |
| 40. | 924.7 | 0.003 | Controls |
| 41. | 926.8 | 0.005 | Controls |
| 42. | 928.8 | 0.006 | Controls |
| 43. | 954.8 | 0.01 | Controls |
| 44. | 956.8 | 0.004 | Controls |
| 45. | 958.8 | 0.005 | Controls |

Replicating serum lipid biomarkers established in second, confirmatory study.

A second confirmation study of the 45 candidate biomarkers was performed to evaluate their performance in a second set of specimens processed and analyzed comparably. This set, also collected at 12-14 wks pregnancy, included 43 controls having uncomplicated term pregnancies and 37 cases having PE later in the same pregnancy. Of the 45 potential biomarkers, 23 continued to show statistically significant or near significant p-values when considered one at a time. These markers are listed Table 3.

TABLE 3
Candidate Biomarkers that Continued to be Statistically
Significant in Second Confirmation Study.

| S. No. | m/z | P value | Higher in |
|---|---|---|---|
| 1. | 263.2 | 0.07 | Cases |
| 2. | 301.2 | 0.005 | Controls |
| 3. | 383.3 | 0.05 | Cases |
| 4. | 425.1 | 7.8 × 10\hat{ }−5 | Controls |
| 5. | 462.3 | 0.04 | Cases |
| 6. | 445.4 | 0.03 | Controls |
| 7. | 645.5 | 0.0001 | Controls |
| 8. | 714.6 | 0.008 | Controls |
| 9. | 734.6 | 0.1 | Cases |
| 10. | 760.6 | 0.06 | Cases |
| 11. | 784.6 | 0.005 | Cases |
| 12. | 788.6 | 0.08 | Controls |
| 13. | 796.6 | 0.06 | Cases |
| 14. | 798.6 | 0.06 | Cases |
| 15. | 810.6 | 0.007 | Cases |
| 16. | 836.6 | 0.06 | Cases |
| 17. | 895.7 | 0.009 | Controls |
| 18. | 916.8 | 0.003 | Controls |
| 19. | 920.8 | 0.003 | Controls |
| 20. | 928.8 | 0.05 | Controls |
| 21. | 954.8 | 0.003 | Controls |
| 22. | 956.8 | 0.003 | Controls |
| 23. | 958.8 | 0.0008 | Controls |

Multi-marker panel development.

Statistical modeling, using logistic regression analysis, was performed on the 23 replicating markers to develop multi-marker panels with higher predictive values. Several panels having combinations of 3-6 markers were obtained demonstrating areas under the curve (AUC) >0.85 for receiver operator characteristic (ROC) curves as illustrated in Table 4.

TABLE 4
Multi-Marker Panels with AUC >0.85.

| AUC | Modeled Marker Combinations |
|---|---|
| 0.89 | 263, 383, 445, 645, 784, 916 |
| 0.89 | 383, 462, 445, 645, 784, 810 |
| 0.89 | 383, 445, 784, 796, 798, 920 |
| 0.88 | 263, 383, 645, 784, 836 |
| 0.86 | 263, 383, 645, 784 |

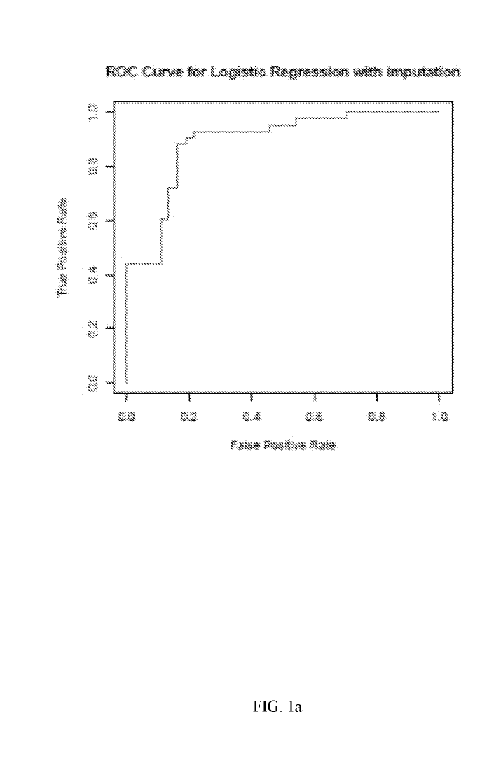

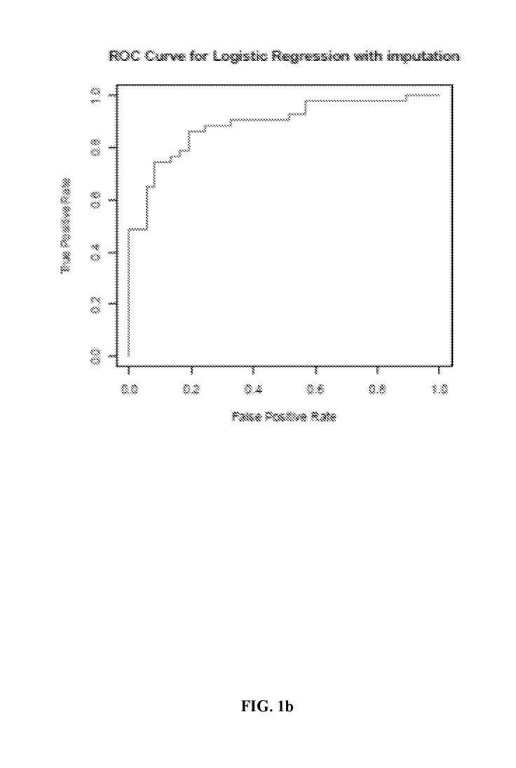

The ROC curves for two multi-marker combinations having AUC of 0.89 (sensitivity ˜91% at a specificity of ˜82% and the second ˜86% and specificity ˜81%) are shown in FIGS. 1a and 1b.

Chemical characterization of the validated serum lipid PE biomarkers.

Though impossible to obtain absolute chemical structures of most lipids using MS, substantial chemical characterization is often provided by tandem MS fragmentation studies. Tandem MS studies were performed on all 23 confirmed serum lipid biomarkers. Table 5 shows exact masses, elemental composition and lipid classes for most of confirmed lipid biomarkers.

TABLE 5
Chemical Characterization of Lipid PE Biomarkers.

| | | Exact | | Elemental | | |
|---|---|---|---|---|---|---|
| No | m/z | mass | Adduct | composition | Class | Possible Identity |
| 1. | 263.2 | 263.23 | M + H⁺ | C₂₀H₂₀O + H⁻ | Fatty alcohol and | |
| | | | | | aldehydes | |
| 2. | 301.2 | 301.21 | M + H⁻ | C₂₀H₂₈O₂ + H⁻ | Fatty acids and | |
| | | | | | conjugates | |
| 3. | 383.3 | 383.33 | M⁻ | C₂₇H₄₃O⁻ | Oxidized cholesterol | 7-keto cholesterol |
| 4. | 452.1 | 425.06 | | | | |
| 5. | 445.4 | 445.36 | M + H⁻ | C₂₉H₄₃O₃ + H⁺ | Cholesterol derivatives | |
| 6. | 462.3 | 462.30 | | | | |
| 7. | 645.5 | 645.55 | M + H⁻ | C₂₅H₇₂O₂ + H⁺ | Cholesterol esters | C18:4 cholesterol |
| | | | | | | ester |
| 8. | 714.6 | 714.61 | M + NH₄⁻ | C₄₉H₂₆O_{2 + NH4}⁺ | Cholesterol esters | C22:6 cholesterol |
| | | | | | | ester |
| 9. | 734.6 | 734.56 | M + H⁺ | c₄₀H₂₀NO₃P + H⁻ | Glycerophosphocholine | PC-16:0/16:0 |
| 10. | 760.6 | 760.58 | M + H⁻ | C₄₂H₈₂NO₃P + H⁺ | Glycerophosphocholine | PC-16:0/18:1 |
| 11. | 784.6 | 784.58 | M + H⁺ | C₄₄H₈₂NO₃P + H⁻ | Glycerophosphocholine | PC-18:1/18:2 |
| 12. | 788.6 | 788.61 | M + H⁻ | C₂₄H₅₅NO₃P + H⁺ | Glycerophosphocholine | PC-18:0/18:1 |
| 13. | 796.6 | 796.62 | M + H⁺ | C₄₂H₂₆NO₃P + H⁻ | Oxidized | PC-O-18:0/20:4 |
| | | | | | Glycerophosphocholine | |
| 14. | 798.6 | 798.56 | M + H⁻ | C₃₄H₂₀NO₉P + H⁻ | Oxidized | PC-(16:0/18:4) + OH |
| | | | | | Glycerophosphocholine | |
| 15. | 810.6 | 810.60 | M + H⁻ | C₄₆H₂₄NO₂P + H⁺ | Glycerophosphocholine | PC-18:0/20:4 |
| 16. | 836.6 | 836.61 | M + H⁻ | C₄₈H₅₆NO₈P + H⁻ | Glycerophosphocholine | PC-18:0/22:5 |
| 17. | 895.7 | 895.72 | M + H⁻ | C₅₂H₉₉N₂O₂P + H⁻ | Oxidized sphingomyclin | |
| 18. | 916.8 | 916.79 | M + NH₄⁺ | C₅₇H₃₀₂O₂ + | Oxidized triacylglycerol | TG-18:0/18:2/18.2 + |
| | | | | NH₄⁻ | | OH |
| 19. | 920.7 | 920.75 | M + NH₄⁺ | C₅₅H₉₅O₉ + NH₄⁺ | Oxidized triacylglycerol | TG-18:2/16:0/18:2 + |
| | | | | | | OH + OOH |
| 20. | 928.8 | 928.76 | M + NH₄⁺ | C₅₇H₉₁O₁ + NH₄⁺ | Oxidized triacylglycerol | TG-18:1/18:1/18:2 + |
| | | | | | | OH + OH |
| 21. | 954.8 | 954.77 | M + NH₄⁺ | C₅₉H₁₀₄O₃ + | Oxidized triacylglycerol | TG-904.75 |
| | | | | NH₄⁻ | | (18:1/18:2/20:4) + |
| | | | | | | OH + OH |
| 22. | 956.8 | 956.79 | M + NH₄⁻ | C₅₉H₁₀₂O₃ + | Oxidized triacylglycerol | TG- |
| | | | | NH₄⁻ | | 906.77(18:0/18:2/20:4) |
| | | | | | | or |
| | | | | | | (18:1/18:1/20:4) + |
| | | | | | | OH + OH |
| 23. | 958.8 | 958.77 | M + NH₄⁻ | C₅₈H₁₀₀O₉ + | Oxidized triacylglycerol | 892.75 + OH + OOH |
| | | | | NH₄⁺ | | |

Fragmentation results with any chemical structural information are summarized briefly for the validated markers:

The biomarker m/z 263.2, when fragmented, demonstrated several hydrocarbon fragments in the low m/z region consistent with its having an alkane region. This marker was found to be a fatty alcohol or aldehyde. The marker with m/z 301.2 showed an elemental composition of C₂₀H₂₈O₂ consistent with its being a fatty acid or fatty acid conjugate. Another marker having m/z 383.3 was found to generate a fragment consistent with its being 7-keto cholesterol (Kemmo et al.; Souidi et al.).

The species at m/z 445.4 was found to be a cholesterol derivative. Fragmentation studies for two other markers with m/z 645.5 and m/z 714.6 resulted in a daughter fragment ion m/z 369.4, characteristic of cholesterol esters, formed with octadecatetraenoic acid (C18:4) and docosahexaenoic acid (22:6) respectively.

Several of the lipid markers were confidently categorized as glycerophosphocholines (PC). The MS-MS fragmentation spectrum for markers with m/z values of 734.6, 760.6, 784.6, 788.6, 796.6, 798.6, 810.6, 836.6 and 895.7 displayed a prominent peak at m/z 184.07 corresponding to a phosphocholine moiety. Their elemental compositions were determined using exact mass studies and the fatty acyl constituents were assigned based on the neutral losses from the parent ion with help from the Lipid MS predictor from Lipid Maps. These same markers (m/z 734.6, 760.6, 784.6, 788.6, 796.6, 810.6, 836.6) were very likely to be PC-16:0/16:0, PC-16:0/18:1, PC-18:1/18:2, PC-O-18:0/20:4, PC-O-18:0/20:4, PC-18:0/20:4 and PC-18:0/22:5 respectively, where PC-O represents an oxidized fatty acyl group. The marker with m/z 798.6 is likely to be an oxidized PC-(16:0/18:4) with a hydroxyl group present. A major neutral water loss peak from the parent, present in the fragmentation spectra for this lipid, indicated an easily removed hydroxyl group.

According to the nitrogen rule, the odd M+H+ (having even neutral mass) should represent an even number of nitrogen atoms in that species. Therefore, the marker with m/z 895.7 is consistent with the presence of 7 oxygens and would represent an oxidized sphingomyelin.

Based on MS-MS fragmentation results, the markers having m/z values of 916.8, 920.7, 928.8, 954.8, 956.8 and 958.8 represented triacylglycerols. Exact mass studies for these markers predicted elemental compositions of C₅₇H₁₀₂O₇+NH₄⁺, C₅₅H₉₈O₉+NH₄⁺, C₅₉H₉₈O₈+NH₄⁺, C₅₉H₁₀₄NO₈+NH₄⁺, C₅₉H₁₀₂O₈+NH₄⁺ and C₅₈H₁₀₀O₈+NH₄⁺ corresponding to oxidized, species, most likely containing a hydroxyl group. These lipids being oxidized was also indicated by neutral water losses from the parent compound during fragmentation.

The markers with m/z 425 and m/z 462 are likely to be lipids but with unknown identities due to fragmentation patterns without precedence in the literature or the database.

Conclusions.

Discussion.

These studies tested the hypothesis that measurement of serum lipid biomarkers early in pregnancy would identify patients at risk for PE later in that pregnancy. Using a ‘global’ or in-depth or ‘shotgun’ serum lipidomics approach, these studies suggest that there are predictive PE biomarkers.

Global serum lipidomic approaches are relatively new and have been applied to only a few clinical indications, e.g. Alzheimer's disease (Han et al. 2011; Han et al. 2007). To our knowledge they have not been previously applied to PE, although there have been a few reports of altered cholesterol and triglyceride profiles in PE (Siddiqui; Aziz et al.; Islam et al.).

Interest in lipids has increased as it has been recognized that lipids are more widely involved in cell regulatory pathways than previously thought. Lipids then may not only be altered in response to disease but it is possible that some circulating or cellular lipid species may mediate or contribute to aspects of disease. Given the uncertainties in the etiology and prediction of PE, there is interest in both developing better assessments of PE risk as well as better understanding the early changes that precede fully manifest PE.

Our approach led to the detection and confirmation of 23 unique lipid biomarkers for PE. It is entirely possible that these circulating factors reflect the consequence of early disease, but some may have a more direct biological role. In the absence of an accepted animal model for PE, early events leading to PE are poorly understood. A number of lipidomic biomarkers identified could be related to pathological processes linked to PE.

Thus, considering the changes in these specific biomarkers may be useful in providing biochemical information about early antecedents of PE. Based on previous literature, some are suggestive. For example, among the several markers, the peak at m/z 383.3, corresponding to 7-ketocholesterol, has been proposed to contribute to atherosclerosis (Rao et al.) and vascular changes similar to atherosclerotic disease have been reported in PE (Staff et al.). This might explain the higher levels of this marker observed in women with later PE.

The candidate biomarkers having m/z 734, 760, 784, 796, 810 and 836 were found to be higher in the serum of preeclamptic women and belong to the lipid class of glycerophosphocholines (PC). Placental ischemia and apoptosis have been reported in PE, resulting in cell lysis with release of membrane constituents, including likely phosphatidylcholines, into the circulation (Neale et al.; Levy). This might explain the higher levels of these markers in PE cases. The marker with m/z 798 likely represents an oxidized (hydroxylated) PC species as indicated by a water loss peak from the parent and the elemental composition (C₄₄H₈₀NO₉P+H⁺) corresponding to one extra oxygen. The exposure of PC species to reactive oxygen species (ROS) can result in oxidation of the species. There have been several reports of increased production of ROS in PE, which might explain the increased production of this marker in PE cases (Packer).

Currently, there are no accepted biomarkers for predicting PE. A number of candidates have been proposed, especially pro-angiogenic and anti-angiogenic factors, and while they have repeatedly demonstrated changes in many women with established PE, they have shown poor sensitivity and specificity in predicting all forms of PE (Kleinrouweler et al.; Powers et al.; Chaiworapongsa et al.). Therefore, there continues to be a need for predictive biomarkers of PE with more reliable sensitivities and specificities. The AUC values of 0.89 for two panels of markers detected in our study are substantially better than the previously reported values in the literature. The utility of these markers across two studies indicates particular utility early in pregnancy for predicting events months later.

Utilization of this direct lipidomic approach as used here may provide a high throughput method for analysis of individual lipid species from diverse classes without a chromatographic separation step. These methods provide comparative quantitation of species, and with appropriate standards can allow for absolute quantitation. The approach allows for chemical characterization of interesting lipids as well as analysis analogous to multiple reaction monitoring as used for peptides and proteins. This method can have a linear range above 1000, even over the low concentration range, making it efficient for studying low abundant lipid species (Han et al. 2005).

### STATEMENTS

The following statements present various additional embodiments of the disclosure:

Statement 1: A method of diagnosing a pregnant subject as having preeclampsia or being at increased risk for preeclampsia, the method comprising analyzing a serum sample from a pregnant subject for levels of one or more lipid markers; and diagnosing the pregnant individual as having preeclampsia or being at increased risk for preeclampsia based on levels of the lipid markers relative to levels in a normal pregnant subject of about the same gestational age.

Statement 2: A method of reducing symptoms or preventing the onset of preeclampsia in a pregnant subject, the method comprising sampling serum from a pregnant individual at about 12-14 weeks gestation; analyzing the sample for levels of three or more lipid markers in the serum; comparing the levels of the three or more lipid markers to levels in control subjects of about the same gestational age, wherein the three or more lipid markers are each separately selected from the group having a mass to charge ratio of about 263.2, 383.3, 445.4, 462.3, 645.5, 784.6, 796.6, 798.6, 810.6, 836.6, 916.8, 920.8, and combinations thereof, as determined by electrospray injection time-of-flight mass-spectrometry; and determining a clinical treatment to reduce symptoms or prevent onset of preeclampsia in the pregnant subject.

Statement 3: The method according to statements 1 or 2, wherein the serum sample is from a pregnant subject at about 12-14 weeks gestation.

Statement 4: The method according to any one of statements 1-3, wherein the at least one lipid marker is detected at least 3 to 6 months prior to a clinical symptom associated with preeclampsia.

Statement 5: The method according to any one of statements 1-4, wherein the pregnant subject exhibits at least 80% sensitivity.

Statement 6: The method according to statement 1, wherein the at least one lipid marker is selected from the group having a mass to charge ratio of about 263.2, 301.2, 383.3, 425.1, 445.4, 462.3, 645.5, 714.6, 734.6, 760.6, 784.6, 788.6, 796.6, 798.6, 810.6, 836.6, 895.7, 916.8, 920.8, 928.8, 954.8, 956.8, 958.8, and combinations thereof, as determined by electrospray injection time-of-flight mass-spectrometry (ESI-TOF MS).

Statement 7: The method according to any one of statements 1-6, wherein pregnant subjects having elevated levels of at least one lipid marker selected from the group having a mass to charge ratio of about 263.2, 383.3, 462.3, 734.6, 760.6, 784.6, 796.6, 798.6, 810.6, 836.6, and combinations thereof, as compared to normal pregnant subjects of about the same gestational age, have an elevated risk of preeclampsia.

Statement 8: The method according to any one of statements 1-6, wherein pregnant subjects having decreased levels of at least one lipid marker selected from the group having a mass to charge ratio of about 301.2, 425.1, 445.4, 645.5, 714.6, 788.6, 895.7, 916.8, 920.8, 928.8, 954.8, 956.8, 958.8, and combinations thereof, as compared to normal pregnant subjects of about the same gestational age, have an elevated risk of preeclampsia.

Statement 9: The method according to statement 1, wherein the at least one lipid marker has an elemental composition selected from the group consisting of C₁₈H₃₀O+H⁺, C₂₀H₂₈O₂+H⁺, C₂₇H₄₃O⁺, C₂₉H₄₈O₃+H⁺, C₄₅H₇₂O₂+H⁺, C₄₉H₇₆O₂+NH⁴⁺, C₄₀H₈₀NO₈P+H⁺, C₄₂H₈₂NO₈P+H⁺, C₄₄H₈₂NO₈P+H⁺, C₄₄N₈₆NO₈P+H⁺, C₄₆H₈₆NO₇P+H⁺, C₄₄H₈₀NO₉P+H⁺, C₄₆H₈₄NO₈P+H⁺, C₄₈H₈₆NO₈P+H⁺, C₅₂H₉₉N₂O₇P+H⁺, C₅₇H₁₀₂O₇+NH⁴⁺, C₅₅H₉₈O₉+NH⁴⁺, C₅₇H₉₈O₈+NH⁴⁺, C₅₉H₁₀₄O₈+NH⁴⁺, C₅₉H₁₀₂O₈+NH⁴⁺C₅₈H₁₀₀O₉+NH⁴⁺, and combinations thereof.

Statement 10: The method according to statement 1, wherein the at least one lipid marker is selected from the group consisting of 7-keto cholesterol, C18:4 cholesterol ester, C22:6 cholesterol ester, PC-16:0/16:0, PC-16:0/18:1, PC-18:1/18:2, PC-18:0/18:1, PC-O-18:0/20:4, PC-(16:0/18:4)+OH, PC-18:0/20:4, PC-18:0/22:5, TG-18:0/18:2/18:2+OH, TG-18:2/16:0/18:2+OH+00H, TG-18:1/18:1/18:2+OH+OH, TG-904.75 (18:1/18:2/20:4)+OH+OH, TG-906.77 (18:0/18:2/20:4)+0H+OH, TG-906.77 (18:1/18:1/20:4)+0H+OH, and combinations thereof.

Statement 11: The method according to statement 1, wherein the serum sample is analyzed for levels of two or more lipid markers selected from the group having a mass to charge ratio of about 263.2, 383.3, 445.4, 462.3, 645.5, 784.6, 796.6, 798.6, 810.6, 836.6, 916.8, 920.8, and combinations thereof, as determined by electrospray injection time-of-flight mass-spectrometry.

Statement 12: The method according to statement 1, wherein the serum sample is analyzed for levels of three or more lipid markers selected from the group having a mass to charge ratio of about 263.2, 383.3, 445.4, 462.3, 645.5, 784.6, 796.6, 798.6, 810.6, 836.6, 916.8, 920.8, and combinations thereof, as determined by electrospray injection time-of-flight mass-spectrometry.

Statement 13: The method according to any one of statements 1-5, wherein the serum sample is analyzed for levels of four or more lipid markers selected from the group having a mass to charge ratio of about 263.2, 383.3, 445.4, 462.3, 645.5, 784.6, 796.6, 798.6, 810.6, 836.6, 916.8, 920.8, and combinations thereof, as determined by electrospray injection time-of-flight mass-spectrometry.

Statement 14: The method according to any one of statements 1-5, wherein the serum sample is analyzed for levels of five or more lipid markers selected from the group having a mass to charge ratio of about 263.2, 383.3, 445.4, 462.3, 645.5, 784.6, 796.6, 798.6, 810.6, 836.6, 916.8, 920.8, and combinations thereof, as determined by electrospray injection time-of-flight mass-spectrometry.

Statement 15: The method according to any one of statements 1-5, further comprising treating the pregnant subject for preeclampsia.

Statement 16: A panel of lipid markers of preeclampsia, the panel comprising at least three lipid markers selected from the group having a mass to charge ratio of about 263.2, 383.3, 445.4, 462.3, 645.5, 784.6, 796.6, 798.6, 810.6, 836.6, 916.8, 920.8, and combinations thereof, as determined by electrospray injection time-of-flight mass-spectrometry.

Statement 17: The panel of statement 16, wherein the lipid markers are obtained from serum of pregnant subjects at about 12-14 weeks gestation.

Statement 18: The panel of statements 16 or 17, comprising the lipid markers having a mass to charge ratio of about 383.3, 645.5, and 784.6.

Statement 19: The panel of statement 18, further comprising the lipid marker having a mass to charge ratio of about 263.2.

Statement 20: The panel of statement 19, further comprising the lipid marker having a mass to charge ratio of about 836.6.

What is claimed is:

1. A method of diagnosing a pregnant subject as having preeclampsia or being at increased risk for preeclampsia, the method comprising:
analyzing a serum sample from a pregnant subject for levels of one or more lipid markers; and
diagnosing the pregnant individual as having preeclampsia or being at increased risk for preeclampsia based on levels of the lipid markers relative to levels in a normal pregnant subject of about the same gestational age.

2. The method of claim 1, wherein the serum sample is from a pregnant subject at about 12-14 weeks gestation.

3. The method of claim 1, wherein the at least one lipid marker is detected at least 3 to 6 months prior to a clinical symptom associated with preeclampsia.

4. The method of claim 1, wherein the pregnant subject exhibits at least 80% sensitivity.

5. The method according to claim 1, wherein the at least one lipid marker is selected from the group having a mass to charge ratio of about 263.2, 301.2, 383.3, 425.1, 445.4, 462.3, 645.5, 714.6, 734.6, 760.6, 784.6, 788.6, 796.6, 798.6, 810.6, 836.6, 895.7, 916.8, 920.8, 928.8, 954.8, 956.8, 958.8, and combinations thereof, as determined by electrospray injection time-of-flight mass-spectrometry (ESI-TOF MS).

6. The method according to claim 5, wherein pregnant subjects having elevated levels of at least one lipid marker selected from the group having a mass to charge ratio of about 263.2, 383.3, 462.3, 734.6, 760.6, 784.6, 796.6, 798.6, 810.6, 836.6, and combinations thereof, as compared to normal pregnant subjects of about the same gestational age, have an elevated risk of preeclampsia.

7. The method according to claim 5, wherein pregnant subjects having decreased levels of at least one lipid marker selected from the group having a mass to charge ratio of about 301.2, 425.1, 445.4, 645.5, 714.6, 788.6, 895.7, 916.8, 920.8, 928.8, 954.8, 956.8, 958.8, and combinations thereof, as compared to normal pregnant subjects of about the same gestational age, have an elevated risk of preeclampsia.

8. The method according to claim 1, wherein the at least one lipid marker has an elemental composition selected from the group consisting of C₁₈H₃₀O+H⁺, C₂₀H₂₈O₂+H⁺, C₂₇H₄₃O⁺, C₂₉H₄₈O₃+H⁺, C₄₅H₇₂O₂+H⁺, C₄₉H₇₆O₂+NH⁴⁺, C₄₀H₈₀NO₈P+H⁺, C₄₂H₈₂NO₈P+H⁺, C₄₄H₈₂NO₈P+H⁺, C₄₄H₈₆NO₈P+H⁺, C₄₆H₈₆NO₇P+H⁺, C₄₄H₈₀NO₉P+H⁺, C₄₆H₈₄NO₈P+H⁺, C₄₈H₈₆NO₈P+H⁺, C₅₂H₉₉N₂O₇P+H⁺, C₅₇H₁₀₂O₇+NH⁴⁺, C₅₅H₉₈O₉+NH⁴⁺, C₅₇H₉₈O₈+NH⁴⁺, C₅₉H₁₀₄O₈+NH⁴⁺, C₅₉H₁₀₂O₈+NH⁴⁺C₅₈H₁₀₀O₉+NH⁴⁺, and combinations thereof.

9. The method according to claim 1, wherein the at least one lipid marker is selected from the group consisting of 7-keto cholesterol, C18:4 cholesterol ester, C22:6 cholesterol ester, PC-16:0/16:0, PC-16:0/18:1, PC-18:1/18:2, PC-18:0/18:1, PC-O-18:0/20:4, PC-(16:0/18:4)+OH, PC-18:0/20:4, PC-18:0/22:5, TG-18:0/18:2/18:2+OH, TG-18:2/16:0/18:2+OH+OOH, TG-18:1/18:1/18:2+OH+OH, TG-904.75 (18:1/18:2/20:4)+OH+OH, TG-906.77 (18:0/18:2/20:4)+OH+OH, TG-906.77 (18:1/18:1/20:4)+OH+OH, and combinations thereof.

10. The method according to claim 1, wherein the serum sample is analyzed for levels of two or more lipid markers selected from the group having a mass to charge ratio of about 263.2, 383.3, 445.4, 462.3, 645.5, 784.6, 796.6, 798.6, 810.6, 836.6, 916.8, 920.8, and combinations thereof, as determined by electrospray injection time-of-flight mass-spectrometry.

11. The method according to claim 1, wherein the serum sample is analyzed for levels of three or more lipid markers selected from the group having a mass to charge ratio of about 263.2, 383.3, 445.4, 462.3, 645.5, 784.6, 796.6, 798.6, 810.6, 836.6, 916.8, 920.8, and combinations thereof, as determined by electrospray injection time-of-flight mass-spectrometry.

12. The method according to claim 1, wherein the serum sample is analyzed for levels of four or more lipid markers selected from the group having a mass to charge ratio of about 263.2, 383.3, 445.4, 462.3, 645.5, 784.6, 796.6, 798.6, 810.6, 836.6, 916.8, 920.8, and combinations thereof, as determined by electrospray injection time-of-flight mass-spectrometry.

13. The method according to claim 1, wherein the serum sample is analyzed for levels of five or more lipid markers selected from the group having a mass to charge ratio of about 263.2, 383.3, 445.4, 462.3, 645.5, 784.6, 796.6, 798.6, 810.6, 836.6, 916.8, 920.8, and combinations thereof, as determined by electrospray injection time-of-flight mass-spectrometry.

14. A method of reducing symptoms or preventing the onset of preeclampsia in a pregnant subject, the method comprising:
sampling serum from a pregnant individual at about 12-14 weeks gestation;
analyzing the sample for levels of three or more lipid markers in the serum;
comparing the levels of the three or more lipid markers to levels in control subjects of about the same gestational age, wherein the three or more lipid markers are each separately selected from the group having a mass to charge ratio of about 263.2, 383.3, 445.4, 462.3, 645.5, 784.6, 796.6, 798.6, 810.6, 836.6, 916.8, 920.8, and combinations thereof, as determined by electrospray injection time-of-flight mass-spectrometry; and
determining a clinical treatment to reduce symptoms or prevent onset of preeclampsia in the pregnant subject.

15. The method according to claim 14, further comprising treating the pregnant subject for preeclampsia.

16. A panel of lipid markers of preeclampsia, the panel comprising at least three lipid markers selected from the group having a mass to charge ratio of about 263.2, 383.3, 445.4, 462.3, 645.5, 784.6, 796.6, 798.6, 810.6, 836.6, 916.8, 920.8, and combinations thereof, as determined by electrospray injection time-of-flight mass-spectrometry.

17. The panel of claim 16, wherein the lipid markers are obtained from serum of pregnant subjects at about 12-14 weeks gestation.

18. The panel of claim 16, comprising the lipid markers having a mass to charge ratio of about 383.3, 645.5, and 784.6.

19. The panel of claim 18, further comprising the lipid marker having a mass to charge ratio of about 263.2.

20. The panel of claim 18, further comprising the lipid marker having a mass to charge ratio of about 836.6.
